(19) United States
(12) Patent Application Publication
(10) Pub. No.: US 2012/0107955 A1
(43) Pub. Date: May 3, 2012

(54) METABOLITE BIOMARKERS FOR THE DETECTION OF ESOPHAGEAL CANCER USING MS

(75) Inventors: M. Daniel Raftery, West Lafayette, IN (US); Danijel Djukovic, Indianapolis, IN (US); Zane Hammoud, Northville, MI (US)

(73) Assignee: Purdue Research Foundation, West Lafayette, IN (US)

(21) Appl. No.: 13/226,236

(22) Filed: Sep. 6, 2011

Related U.S. Application Data
(60) Provisional application No. 61/402,731, filed on Sep. 3, 2010.

(51) Int. Cl. G01N 33/574; C07H 19/067; G01N 21/00; C07H 19/167

(52) U.S. Cl. 436/501; 536/27.6; 536/27.81; 536/28.5; 536/28.53; 252/408.1

(57) ABSTRACT

Results of studies of nucleosides in biofluid specimens from patients with esophageal adenocarcinoma and related disorders have identified five biomarkers of the conditions: 1-methyladenosine, N²,N²-dimethylguanosine, N²-methylguanosine, cytidine and uridine. In certain embodiments, methods of measuring these biomarkers and kits for measuring these biomarkers are provided.

### RELATED APPLICATION

This application claims benefit of U.S. Provisional Patent Application 61/402,731, filed Sep. 3, 2010, the entire contents of which are incorporated by reference for all purposes.

### TECHNICAL FIELD

The present disclosure generally relates to small molecule biomarkers comprising a set of metabolite species that is effective for the early detection of esophageal cancer, including methods for identifying such biomarkers within biological samples.

### BACKGROUND

Esophageal cancer is a leading cause of death from cancer worldwide. The two principal types of esophageal cancer are squamous cell carcinoma and adenocarcinoma. Both are relatively uncommon in the U.S., comprising approximately 1% of all cancers. However, the incidence of adenocarcinoma is rising at a rapid rate. According to a report from American Cancer Society, 12,300 new cases and 12,100 deaths were reported in 2000, and the corresponding numbers for 2009 are 16,470 and 14,530, respectively. The 5-year survival rates for localized and all stages combined are 34% and 17%, respectively. Moreover, there is no currently reliable method for early detection or for the prediction of treatment outcome.

Barrett's esophagus (BE), high-grade dysplasia (HGD), and invasive cancer are thought to comprise a multi-step process in the development of esophageal adenocarcinoma (EAC). HGD has been considered as the immediate precursor of invasive adenocarcinoma. Since most patients with HGD are usually bearing or developing cancer, HGD has been regarded as a marker of progression to carcinoma. However, no intervention currently exists that prevents the progression of BE or HGD to esophageal cancer. The traditional methods for diagnosing esophageal cancer include endoscopy and barium swallow, but the poor specificity and sensitivity of these methods results in their detection only at an advanced stage. Recently, prognostic and predictive protein and genetic markers have been introduced to aid in the diagnosis of esophageal cancer. However, markers effective at a potentially curative stage are lacking.

Metabolomics (or metabolite profiling) is the study of concentrations and fluxes of low molecular weight metabolites present in biofluids or tissues that provide detailed information on biological systems and their current status. The field of metabolomics emphasizes the multiplexed analysis of known and unknown metabolites in complex biological matrices such as pathological and normal tissue and biological fluids. Metabolomics aims to improve the molecular level understanding of metabolic pathways associated with many diseases or other biological states in a system biology approach.

Nucleosides are building blocks of ribonucleic acid (RNA) and their excretion is an indicator of the whole-body turnover of RNA. Nucleosides from RNA structurally consist of a nucleobase (nitrogenous base) bound to a ribose sugar through the N-glycosidic bond. RNA contains the four major nucleosides (cytidine, uridine, guanosine and adenosine) and a number of modified nucleosides that are methylated analogues of these four. The methylation usually occurs at nitrogen sites in the nucleobase part of the molecule. Of all forms of RNA, transfer RNA (tRNA) contains the largest number of known modified nucleosides. Studies have suggested that nucleosides are elevated in cancer tissues and are excreted at an increased level with malignancy. Increased concentrations of nucleosides have been found in lung, ovarian, colorectal, breast, bladder, liver and thyroid cancer patients. Abnormally high concentrations of nucleosides were also reported in association with leukemia, lymphoma, and Hodgkin's disease.

Early diagnostic methods that offer high sensitivity and specificity for detecting esophageal cancer are in great demand.

### SUMMARY OF THE INVENTION

Results studies of nucleosides in biofluid specimens from patients with esophageal adenocarcinoma and related disorders have identified five biomarkers of the conditions: 1-methyladenosine, N²,N²-dimethylguanosine, N²-methylguanosine, cytidine and uridine. In certain embodiments, methods of measuring these biomarkers and kits for measuring these biomarkers are provided.

The present disclosure provides a method for determining the esophageal cancer status of a patient, comprising: determining the presence and concentration within a biological sample from the patient of at least one compound selected from the group consisting of 1-methyl-adenosine, N²,N²-dimethylguanosine, N²-methylguanosine, cytidine, uridine and combinations thereof; and correlating the measured concentration of the metabolite species with an esophageal cancer status. In certain embodiments, the combination of metabolite species is selected from the group consisting of 1-methyladenosine and N²,N²-dimethylguanosine; 1-methyladenosine, N²,N²-dimethylguanosine, N²-methylguanosine, cytidine, and uridine; 1-methyladenosine, N²,N²-dimethylguanosine, and N²-methylguanosine; 1-methyladenosine, N²,N²-dimethylguanosine, N²-methylguanosine, and uridine; 1-methyladenosine, N²,N²-dimethylguanosine, N²-methyl-guanosine, and cytidine; 1-methyladenosine, N²,N²-dimethylguanosine, and uridine; N²,N²-dimethylguanosine, N²-methylguanosine, cytidine, and uridine; and N²,N²-dimethylguanosine, N²-methylguanosine, and uridine. The esophageal cancer status can be normal, Barrett's esophagus, high-grade dysplasia or esophageal adenocarcinoma. Typically, the sample comprises a biofluid, such as blood or serum.

Typically the metabolite species are adapted to function as biomarkers. In certain embodiments, a biomarker is useful for detecting esophageal cancer, comprising at least one metabolite species or parts thereof, selected from the group consisting of 1-methyladenosine, N²,N²-dimethylguanosine, N²-methylguanosine, cytidine, uridine and combinations thereof. In certain embodiments, the combination of metabolite species is selected from the group consisting of 1-methyladenosine and N²,N²-dimethylguanosine; 1-methyladenosine, N²,N²-dimethylguanosine, N²-methylguanosine, cytidine, and uridine; 1-methyladenosine, N²,N²-dimethylguanosine, and N²-methylguanosine; 1-methyladenosine, N², N²-dimethylguanosine, N²-methylguanosine, and uridine; 1-methyladenosine, N²,N²-dimethylguanosine, N²-methyl-guanosine, and cytidine; 1-methyladenosine, N²,N²-dimethylguanosine, and uridine; N²,N²-dimethylguanosine, N²-methylguanosine, cytidine, and uridine; and N²,N²-dimethylguanosine, N²-methylguanosine, and uridine. The esophageal cancer status can be normal, Barrett's esophagus, high-grade dysplasia or esophageal adenocarcinoma. Typically, the biomarker is used in a biochemically compatible solution, such as a compatible buffered aqueous solution.

In other embodiments, a kit for the analysis of a sample of a biofluid of a subject is provided, comprising standard aliquots of 1-methyladenosine, N²,N²-dimethylguanosine, N²-methylguanosine, cytidine, and uridine; an aliquot of an internal standard; and an aliquot of a control biofluid. Typically, the biofluid is serum. In certain embodiments, the internal standard is 7-deaza-adenosine. Typically, the kit includes instructions for use.

### BRIEF DESCRIPTION OF THE DRAWINGS

The above-mentioned aspects of the present teachings and the manner of obtaining them will become more apparent and the teachings will be better understood by reference to the following description of the embodiments taken in conjunction with the accompanying drawings, in which corresponding reference characters indicate corresponding parts throughout the several views:

FIG. 1 is a schematic diagram of the HPLC-triple-quadrupole MS (“HPLC/TQMS”) system used to quantify metabolic levels of modified nucleosides in serum;

FIG. 2 is a graphical representation of the mean metabolic levels of five nucleosides that were significantly different in serum samples from EAC patients compared to control samples;

FIG. 3A-FIG. 3E is a series of HPLC-TQMS chromatograms of 1-methyladenosine (FIG. 3A), N²,N²-dimethylguanosine (FIG. 3B), N²-methylguanosine (FIG. 3C), cytidine (FIG. 3D) and uridine (FIG. 3E) comparing results from serum samples from EAC patients (continuous line) and those from serum samples from control subjects; and

FIG. 4A-FIG. 4E shows graphical representations of the concentrations of five metabolites, 1-methyladenosine (FIG. 4A), N²,N²-dimethylguanosine (FIG. 4B), N²-methyl-guanosine (FIG. 4C), cytidine (FIG. 4D) and uridine (FIG. 4E), with each result plotted individually by the age of each patient. The majority of the patients were male, the asterisks indicate the data from the two female patients.

### DETAILED DESCRIPTION OF PREFERRED EMBODIMENTS

The present disclosure is directed to the detection and screening of esophageal adenocarcinoma (EAC) patients and to the monitoring of EAC treatment using a panel or panels of small molecule metabolite biomarkers. The biomarkers are sensitive and specific for the detection of EAC, and can be used to classify Barrett's esophagus (BE) and high-grade dysplasia (HGD), which are widely regarded as precursors of EAC.

Nucleosides are indicators of the whole-body turnover of transfer RNA, and thus have potential utility as cancer biomarkers. Several studies have reported that the metabolic levels of some nucleosides can be significantly altered in EAC patients compared to control groups.

Here we report the results of a targeted metabolite investigation of serum nucleosides in esophageal adenocarcinoma specimens. We quantified eight nucleosides using HPLC-TQMS and determined that the metabolic levels of 1-methyladenosine (p<2.14×10⁻⁷), N²,N²-dimethyl-guanosine (p<2.78×10⁻⁷), N²-methylguanosine (p<2.48×10⁻⁶) and cytidine (p<6.98×10⁻⁴) were significantly elevated while the concentration of uridine (p<3.74×10⁻³) was significantly lowered in serum samples from EAC patients compared to those of control group. Our results confirm that nucleosides can serve as useful biomarkers to identify esophageal adenocarcinoma and related conditions.

MS-based metabolite profiling analysis is shown to be an effective approach for differentiating EAC patients and healthy subjects. Good sensitivity and selectivity was shown using metabolite markers discovered to predict the classification of healthy control and disease samples. These markers can be measured using the MS methods described in this application, or using NMR, as described in published patent application WO 2011/046597, the commonly owned and co-pending application entitled “Metabolite Biomarkers for the Detection of Esophageal Cancer using NMR,” docket number 308258.3000-102, filed on even date herewith, or in PCT/US2011/029681. The biomarkers disclosed herein can be adapted for use on various diagnostics workstations or platforms in different formats (clinical chemistry, immunoassay, etc). Suitable analytical techniques for the identification and quantification of these metabolites include immunoassays, capillary electrophoresis, high performance liquid chromatography (HPLC), HPLC coupled to mass spectrometry (MS), and gas chromatography (GC) coupled to MS.

Unless defined otherwise, all technical and scientific terms used herein have the meaning commonly understood by a person skilled in the art to which this invention belongs. Numbers in scientific notation are expressed as product of a coefficient between 1 and 10 and ten raised to an integer power (e.g., 9.6×10⁻⁴), or abbreviated as the coefficient followed by “E,” followed by the exponent (e.g., 9.6E-04).

The terms “first,” “second,” and the like herein do not denote any order or importance, but rather are used to distinguish one element from another, and the terms “a” and “an” herein do not denote a limitation of quantity, but rather denote the presence of at least one of the referenced items. Similarly, it is noted that the terms “bottom” and “top” are used herein, unless otherwise noted, merely for convenience of description, and are not limited to any one position or spatial orientation. In addition, the modifier “about” used in connection with a quantity is inclusive of the stated value and has the meaning dictated by the context (e.g., includes the degree of error associated with measurement of the particular quantity).

As used herein, “metabolite” refers to any substance produced or used during all the physical and chemical processes within the body that create and use energy, such as: digesting food and nutrients, eliminating waste through urine and feces, breathing, circulating blood, and regulating temperature. The term “metabolic precursors” refers to compounds from which the metabolites are made. The term “metabolic products” refers to any substance that is part of a metabolic pathway (e.g. metabolite, metabolic precursor).

As used herein, “biological sample” refers to a sample obtained from a subject. In preferred embodiments, biological sample can be selected, without limitation, from the group of biological fluids (“biofluids”) consisting of blood, plasma, serum, sweat, saliva, including sputum, urine, and the like. As used herein, “serum” refers to the fluid portion of the blood obtained after removal of the fibrin clot and blood cells, distinguished from the plasma in circulating blood. As used herein, “plasma” refers to the fluid, non-cellular portion of the blood, as distinguished from the serum, which is obtained after coagulation.

As used herein, “subject” refers to any warm-blooded animal, particularly including a member of the class Mammalia such as, without limitation, humans and non-human primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex and, thus, includes adult and newborn subjects, whether male or female. As used herein, “normal control subjects” or “normal controls” means healthy subjects who are clinically free of cancer. “Normal control sample” or “control sample” refers to a sample of biofluid that has been obtained from a normal control subject.

As used herein, “detecting” refers to methods which include identifying the presence or absence of substance(s) in the sample, quantifying the amount of substance(s) in the sample, and/or qualifying the type of substance. “Detecting” likewise refers to methods which include identifying the presence or absence of BE, HGD and EAC or the progression of EAC.

“Mass spectrometer” refers to a gas phase ion spectrometer that measures a parameter that can be translated into mass-to-charge ratios of gas phase ions. Mass spectrometers generally include an ion source and a mass analyzer. Examples of mass spectrometers are time-of-flight, magnetic sector, quadrupole filter, ion trap, ion cyclotron resonance, electrostatic sector analyzer and hybrids of these. “Mass spectrometry” refers to the use of a mass spectrometer to detect gas phase ions.

It is to be understood that this invention is not limited to the particular component parts of a device described or process steps of the methods described, as such devices and methods may vary. It is also to be understood that the terminology used herein is for purposes of describing particular embodiments only, and is not intended to be limiting. As used in the specification and the appended claims, the singular forms “a,” “an,” and “the” include plural referents unless the context clearly indicates otherwise. The terms “comprises,” “comprising,” and the like are intended to have the broad meaning ascribed to them in U.S. Patent Law and can mean “includes,” “including” and the like.

Metabolite profiling uses high-throughput analytical methods such as nuclear magnetic resonance spectroscopy and mass spectroscopy for the quantitative analysis of hundreds of small molecules (less than ˜1000 Daltons) present in biological samples. Owing to the complexity of the metabolic profile, multivariate statistical methods are extensively used for data analysis. The high sensitivity of metabolite profiles to even subtle stimuli can provide the means to detect the early onset of various biological perturbations in real time.

While these metabolite profiles were discovered using platforms of NMR and LC-MS methods, one of ordinary skill in the art will recognize that these identified biomarkers can be detected by alternative methods of suitable sensitivity, such as HPLC, immunoassays, enzymatic assays or clinical chemistry methods.

In one embodiment of the invention, samples may be collected from individuals over a longitudinal period of time. Obtaining numerous samples from an individual over a period of time can be used to verify results from earlier detections and/or to identify an alteration in marker pattern as a result of, for example, pathology. In preferred embodiments, the present disclosure provides methods of monitoring the progression of BE, HGD and EAC. In certain embodiments, the present disclosure provides methods of assessing the effectiveness of the treatment of BE, HGD and EAC.

In one embodiment of the invention, the samples are analyzed without additional preparation and/or separation procedures. In another embodiment of the invention, sample preparation and/or separation can involve, without limitation, any of the following procedures, depending on the type of sample collected and/or types of metabolic products searched: removal of high abundance polypeptides (e.g., albumin, and transferrin); addition of preservatives and calibrants, desalting of samples; concentration of sample substances; protein digestions; and fraction collection. In yet another embodiment of the invention, sample preparation techniques concentrate information-rich metabolic products and deplete polypeptides or other substances that would carry little or no information such as those that are highly abundant or native to serum.

In another embodiment of the invention, sample preparation takes place in a manifold or preparation/separation device. Such a preparation/separation device may, for example, be a microfluidics device, such as a cassette. In yet another embodiment of the invention, the preparation/separation device interfaces directly or indirectly with a detection device. Such a preparation/separation device may, for example, be a fluidics device.

In another embodiment of the invention, the removal of undesired polypeptides (e.g., high abundance, uninformative, or undetectable polypeptides) can be achieved using high affinity reagents, high molecular weight filters, column purification, ultracentrifugation and/or electrodialysis. High affinity reagents include antibodies that selectively bind to high abundance polypeptides or reagents that have a specific pH, ionic value, or detergent strength. High molecular weight filters include membranes that separate molecules on the basis of size and molecular weight. Such filters may further employ reverse osmosis, nanofiltration, ultrafiltration and microfiltration.

Ultracentrifugation constitutes another method for removing undesired polypeptides. Ultracentrifugation is the centrifugation of a sample at about 60,000 rpm while monitoring with an optical system the sedimentation (or lack thereof) of particles. Finally, electrodialysis is an electromembrane process in which ions are transported through ion permeable membranes from one solution to another under the influence of a potential gradient. Since the membranes used in electrodialysis have the ability to selectively transportions having positive or negative charge and reject ions of the opposite charge, electrodialysis is useful for concentration, removal, or separation of electrolytes.

In another embodiment of the invention, the manifold or microfluidics device performs electrodialysis to remove high molecular weight polypeptides or undesired polypeptides. Electrodialysis can be used first to allow only molecules under approximately 3530 kD to pass through into a second chamber. A second membrane with a very small molecular weight cutoff (roughly 500 D) allows smaller molecules to exit the second chamber.

Upon preparation of the samples, metabolic products of interest may be separated in another embodiment of the invention. Separation can take place in the same location as the preparation or in another location. In one embodiment of the invention, separation occurs in the same microfluidics device where preparation occurs, but in a different location on the device. Samples can be removed from an initial manifold location to a microfluidics device using various means, including an electric field. In another embodiment of the invention, the samples are concentrated during their migration to the microfluidics device using reverse phase beads and an organic solvent elution such as 50% methanol. This elutes the molecules into a channel or a well on a separation device of a microfluidics device.

Chromatography constitutes another method for separating subsets of substances. Chromatography is based on the differential absorption and elution of different substances. Liquid chromatography (LC), for example, involves the use of fluid carrier over a non-mobile phase. Conventional LC columns have an in inner diameter of roughly 4.6 mm and a flow rate of roughly 1 ml/min. Micro-LC has an inner diameter of roughly 1.0 mm and a flow rate of roughly 40 μl/min. Capillary LC utilizes a capillary with an inner diameter of roughly 300 μm and a flow rate of approximately 5 μl/min. Nano-LC is available with an inner diameter of 50 μm-1 mm and flow rates of 200 nl/min. The sensitivity of nano-LC as compared to HPLC is approximately 3700 fold. Other types of chromatography suitable for additional embodiments of the invention include, without limitation, thin-layer chromatography (TLC), reverse-phase chromatography, high-performance liquid chromatography (HPLC), and gas chromatography (GC).

In another embodiment of the invention, the samples are separated using capillary electrophoresis separation. This will separate the molecules based on their electrophoretic mobility at a given pH (or hydrophobicity). In another embodiment of the invention, sample preparation and separation are combined using microfluidics technology. A microfluidic device is a device that can transport liquids including various reagents such as analytes and elutions between different locations using microchannel structures.

Suitable detection methods are those that have a sensitivity for the detection of an analyte in a biofluid sample of at least 500 μM, more preferably at least 50 μM. In certain embodiments, the sensitivity of the detection method is at least 1 μM. In other embodiments, the sensitivity of the detection method is at least 10 nM.

In one embodiment of the invention, the sample may be delivered directly to the detection device without preparation and/or separation beforehand. In another embodiment of the invention, once prepared and/or separated, the metabolic products are delivered to a detection device, which detects them in a sample. In another embodiment of the invention, metabolic products in elutions or solutions are delivered to a detection device by electrospray ionization (ESI). In yet another embodiment of the invention, nanospray ionization (NSI) is used. Nano spray ionization is a miniaturized version of ESI and provides low detection limits using extremely limited volumes of sample fluid.

In another embodiment of the invention, separated metabolic products are directed down a channel that leads to an electrospray ionization emitter, which is built into a microfluidic device (an integrated ESI microfluidic device). Such integrated ESI microfluidic device may provide the detection device with samples at flow rates and complexity levels that are optimal for detection. Furthermore, a microfluidic device may be aligned with a detection device for optimal sample capture.

Suitable detection devices can be any device or experimental methodology that is able to detect metabolic product presence and/or level, including, without limitation, IR (infrared spectroscopy), NMR (nuclear magnetic resonance), including variations such as correlation spectroscopy (COSy), nuclear Overhauser effect spectroscopy (NOESY), and rotating frame nuclear Overhauser effect spectroscopy (ROESY), and Fourier Transform, 2-D PAGE technology, Western blot technology, tryptic mapping, in vitro biological assay, immunological analysis, LC-MS (liquid chromatography-mass spectrometry), LC-TOF-MS, LC-MS/MS, and MS (mass spectrometry).

For analysis relying on the application of NMR spectroscopy, the spectroscopy may be practiced as one-, two-, or multidimensional NMR spectroscopy or by other NMR spectroscopic examining techniques, among others also coupled with chromatographic methods (for example, as LC-NMR). In addition to the determination of the metabolic product in question, ¹H-NMR spectroscopy offers the possibility of determining further metabolic products in the same investigative run. Combining the evaluation of a plurality of metabolic products in one investigative run can be employed for so-called “pattern recognition”. Typically, the strength of evaluations and conclusions that are based on a profile of selected metabolites, i.e., a panel of identified biomarkers, is improved compared to the isolated determination of the concentration of a single metabolite.

For immunological analysis, for example, the use of immunological reagents (e.g. antibodies), generally in conjunction with other chemical and/or immunological reagents, induces reactions or provides reaction products which then permit detection and measurement of the whole group, a subgroup or a subspecies of the metabolic product(s) of interest. Suitable immunological detection methods with high selectivity and high sensitivity (10-1000 pg, or 0.02-2 pmoles), e.g., Baldo, B. A., et al. 1991, A Specific, Sensitive and High-Capacity Immunoassay for PAF, Lipids 26(12): 1136-1139), that are capable of detecting 0.5-21 ng/ml of an analyte in a biofluid sample (Cooney, S. J., et al., Quantitation by Radioimmunoassay of PAF in Human Saliva), Lipids 26(12): 1140-1143).

In one embodiment of the invention, mass spectrometry is relied upon to detect metabolic products present in a given sample. In another embodiment of the invention, an ESI-MS detection device. Such an ESI-MS may utilizes a time-of-flight (TOF) mass spectrometry system. Quadrupole mass spectrometry, ion trap mass spectrometry, and Fourier transform ion cyclotron resonance (FTICR-MS) are likewise contemplated in additional embodiments of the invention.

In another embodiment of the invention, the detection device interfaces with a separation/preparation device or microfluidic device, which allows for quick assaying of many, if not all, of the metabolic products in a sample. A mass spectrometer may be utilized that will accept a continuous sample stream for analysis and provide high sensitivity throughout the detection process (e.g., an ESI-MS). In another embodiment of the invention, a mass spectrometer interfaces with one or more electrosprays, two or more electrosprays, three or more electrosprays or four or more electrosprays. Such electrosprays can originate from a single or multiple microfluidic devices.

In another embodiment of the invention, the detection system utilized allows for the capture and measurement of most or all of the metabolic products introduced into the detection device. In another embodiment of the invention, the detection system allows for the detection of change in a defined combination (“profile,” “panel,” “ensemble, or “composite”) of metabolic products.

### Working Examples

The metabolic levels of ten nucleosides in serum samples collected from esophageal adenocarcinoma patients and healthy individuals was examined using HPLC-triple-quadrupole MS (“TQMS”). The metabolic levels of 1-methyladenosine, N²,N²-dimethylguanosine, N²-methylguanosine and cytidine were significantly elevated, whereas the concentration of uridine was significantly lower in EAC patients compared to healthy individuals. The role of these metabolites in the disease will be discussed below.

Chemicals All ten standard nucleoside samples (N²,N²-dimethylguanosine, N²-methyl-guanosine, 1-methyladenosine, uridine, cytidine, guanosine, inosine, 5-methylcytidine, 5-hydroxymethyl-2′-deoxyuridine and 8-hydroxy-2′-deoxyguanosine) and 7-deazaadenosine (tubercidin, used as an internal standard, “iSTD”) were purchased from Sigma-Aldrich (St. Louis, Mo.). According to the manufacturer, the purity of each standard was ≧98%. HPLC-grade methanol (MeOH), ammonium acetate, and acetic acid (AcOH) were obtained from Fisher Scientific (Pittsburgh, Pa.). Water was dispensed from an EASYpure II UV water purification system (Barnstead International, Dubuque, Iowa).

Standard Solutions and Calibration Curves Eleven 100 μM stock standard solutions were prepared separately by dissolving each of the foregoing nucleosides and iSTD in a solution composed of 5 mM ammonium acetate in 90% H₂O, 10% MeOH, and 0.1% AcOH. The stock solutions of the nucleosides were then diluted at various concentrations. The concentration range used for the calibration curves was 0.100 μM (0.100 nmol/mL) to 10.0 μM. The prepared samples were kept at −20° C. until analysis. Just prior to the HPLC-TQMS injection, each standard solution was spiked with the internal standard.

The final concentration of iSTD in the standard solutions was 0.500 μM. Since the addition of iSTD solution to the standard samples slightly altered the concentrations of the nucleosides (10 μL of iSTD per 1 mL of standard solution), their concentration values were recalculated. Ten calibration curves (concentration of standard nucleoside vs. peak area/iSTD peak area), one for each nucleoside, were plotted using six data points, and they all showed a very good linearity with r² values ranging from 0.996 to 0.999. The calibration curves were later used to quantify nucleosides in the serum samples.

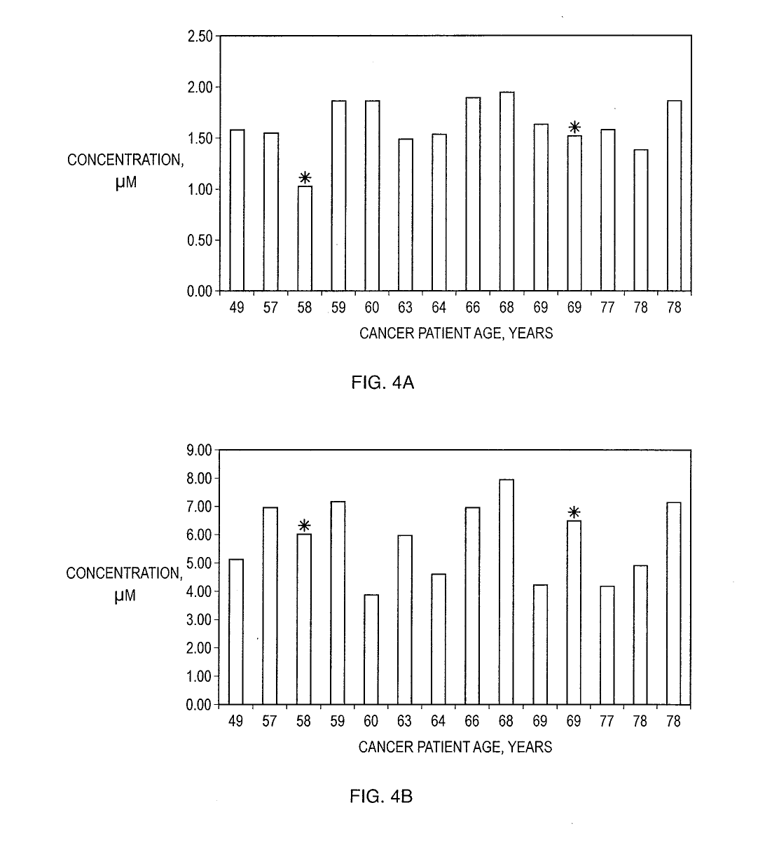

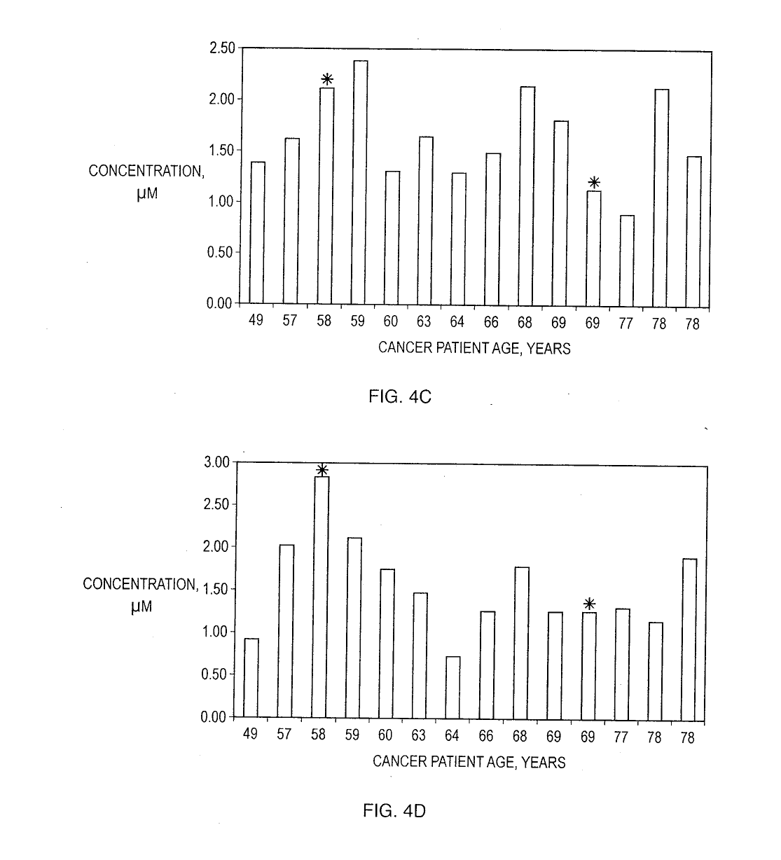

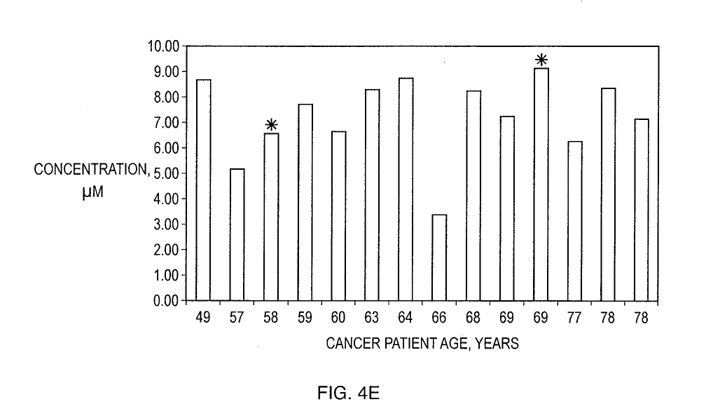

Sample Collection Samples were collected at Indiana University Hospital following Indiana University Hospital and Purdue University Institutional Review Board-approved protocols. All subjects included in the study provided informed consent according to institutional guidelines. Whole blood samples were collected in the fasting state from patients suffering from various stages of esophageal adenocarcinoma (n=14) and from healthy adult volunteers (control samples, n=12). Each blood sample was allowed to clot for 45 min, and then centrifuged at 2000 rpm for 10 min., after which the serum supernatant was aspirated. The serum samples were stored at −80° C. The EAC patient samples for this study were collected from 12 male patients (49, 57, 59, 60, 63, 64, 68, 69, 77, 78, and 78 years of age) and 2 female patients (58 and 69 years of age, indicated by asterisks in FIG. 4A-FIG. 4E). The clinical stages were T1N0 (n=2), T3N1 (n=6), T4N1 (n=3), and stage 4 (n=3). None of the patients received any therapy prior to specimen collection. The mean patient age was 65.4±8.6. No age or gender information was available for the 12 control samples.

Sample Preparation Preparation of serum samples consisted of protein precipitation and solid phase extraction (SPE).

Protein Precipitation: 200 μL methanol was added to 100 μL serum, and the resulting solution was mixed for 1 min. and then left for 20 min. at −20° C. Afterwards, the sample was centrifuged (Eppendorf model 5804, Hauppauge, N.Y.) at 10,000 rpm for 20 min., and the supernatant was collected and dried utilizing a Vacufuge Plus System (Eppendorf). Dried residue was then reconstituted in 1.00 mL of 5 mM ammonium acetate in 99.9% H₂O/0.1% AcOH.

Solid Phase Extraction: In order to extract and concentrate nucleosides from previously purified serum samples, solid phase extraction was performed on Oasis MCX SPE cartridges (Waters Corporation, Milford, Mass.) using a 12-position vacuum manifold (Phenomenex, Torrance, Calif.). The cartridge was conditioned with MeOH and H₂O, 1.00 mL each. 1.00 mL of the sample was loaded onto the cartridge. Afterwards, the cartridge was washed with 1.00 mL 0.1% AcOH in H₂O, and the sample was eluted with 1.00 mL 2.8% NH₄OH in MeOH. The elution solvent was dried and the sample was reconstituted in a 40.0 μL solution of 5 mM ammonium acetate in 90% H₂O, 10% MeOH, and 0.1% AcOH. Each sample was spiked with iSTD. The final concentration of iSTD in each serum sample was 0.500 μM. Since the initial and final sample volumes were 100 μL and 40.0 respectively, a concentration factor of 2.5-fold was used in calculating the concentrations of nucleosides in the serum samples. The prepared samples were kept at −20° C. until analysis. EAC and control samples were placed in random order in the autosampler and were kept at 4° C. throughout analysis.

Instrument Description The analysis of standard and serum samples were performed using HPLC-TQMS. The HPLC-TQMS system included two 1100 Series quaternary pumps equipped with degassers (Agilent Technologies, Santa Clara, Calif.); a temperature controlled CTC PAL autosampler (LEAP Technologies, Carrboro, N.C.); an 1100 Series temperature controlled column oven (Agilent Technologies); a VIC₁₋₂-position switching valve (Valco Instruments, Houston, Tex.); and a Sciex API3000 TQMS equipped with heated Turbo IonSpray source (Applied Biosystems, Streetsville, ON, CA). All the components were controlled in fully automated mode using Analyst 1.5 software (Applied Biosystems).

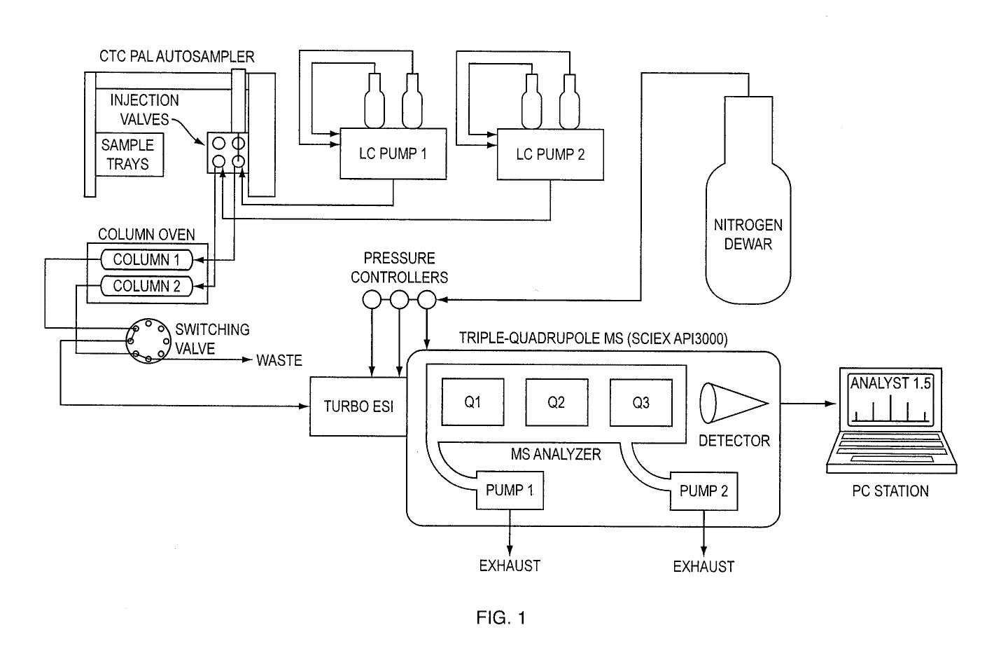

FIG. 1 is a schematic of the HPLC/TQMS system used to quantify metabolic levels of modified nucleosides in serum. Since a limited number of nucleosides (10) and samples (n=26 total) were analyzed, a single analytical column was used. However, as shown in FIG. 1, the system is designed to use two columns connected to two separate injection ports. While analytical separation is performed on one column, the other column is conditioned. There are two main advantages of this instrumental design: 1) it provides faster throughput; and 2) it allows use of two different analytical columns, so it is possible to separate a broader collection of molecules that could not efficiently be separated on a single column. This would especially be useful when analyzing compounds belonging to different chemical classes of metabolites.

Chromatography Conditions Chromatography was performed on an Atlantis dC18 reverse phase column (Waters Corporation, Milford, Mass.). The injection volume and flow rate were 10 μL and 150 μL/min, respectively. The mobile phase consisted of two solvents: (A) 2 mM ammonium acetate in 90% H₂O/10% MeOH/0.1% AcOH, and (B) 2 mM ammonium acetate in 10% H₂O/90% MeOH/0.1% AcOH. The following gradient conditions were used: 90% A/10% B (5 min.)→90% A/10% B to 50% A/50% B (15 min.)→50% A/50% B (5 min.)→50% A/50% B to 90% A/10% B (10 min.)→90% A/10% B (5 min.). The total HPLC/TQMS run time was 40 min. Column conditioning was performed after each sample run.

MS Conditions Electro-spray ionization (“ESI”) was performed in positive ion-mode by applying a voltage of 4 kV. The capillary temperature in the ESI source was 300° C. Nitrogen gas was used as a nebulizer, curtain, and collusion gas at the flow rates of 10, 8, and 10 (arbitrary units), correspondingly. MS acquisition was performed in multiple reaction-monitoring mode (“MRM”). For the MS/MS analysis using a triple quadrupole mass spectrometer running in MRM mode, each nucleoside was first filtered through Q1 as protonated precursor ion, [MH]+. In Q2 (collision cell), [MH]⁺ lost either the ribose sugar to form the [MH-132]⁺ daughter ion or the 2′-deoxyribose moiety to form the [MH-116]⁺ fragment ion. The fragment ion was then filtered through Q3. The optimization of MRM conditions for each nucleoside was performed by directly infusing 2 μM standard solutions at a flow rate of 20 μL/min. The values of de-clustering, focusing, entrance, collision energy and collision cell exit potentials were carefully optimized in order to obtain the most intensive and stable MRM signal for each metabolite. Precursor/daughter ion-transition pairs for the ten nucleosides and internal standard (iSTD) as well as their MRM optimized conditions are shown in Table 1, below. Each MS acquisition for the serum samples simultaneously monitored for all 11 parent/daughter ion pairs (10 nucleosides plus iSTD). Each pair was scanned for 50 ms. The peak area of each nucleoside was referenced to the peak area of the iSTD (tubercidin). The final concentrations of each nucleoside in the EAC and control samples were determined using the standard calibration curves as described above.

TABLE 1
LIST OF THE NUCLEOSIDES' ION-TRANSITION PAIRS
AND THEIR OPTIMIZED MS CONDITIONS IN MRM MODE

| | [M − H]⁺ | [B − H]⁺ | DP^{a} | FP^{b} | EP^{c} | CE^{d} | CXP^{e} |
|---|---|---|---|---|---|---|---|
| COMPOUND | (Q1) | (Q3) | (eV) | (eV) | (eV) | (eV) | (eV) |
| 7 deaza-adenosine (tubercidin, iSTD) | 267.3 | 135.2 | 30 | 200 | 10 | 28 | 11 |
| N²,N²-Dimethylguanosine | 312.3 | 180.2 | 22 | 180 | 10 | 19 | 15 |
| N²-Methylguanosine | 298.3 | 166.2 | 15 | 150 | 10 | 16 | 15 |
| 1-Methyladenosine | 282.3 | 150.2 | 28 | 200 | 8 | 25 | 14 |
| Uridine | 245.2 | 113.1 | 42 | 180 | 9 | 14 | 15 |
| Cytidine | 244.2 | 112.1 | 30 | 200 | 10 | 26 | 10 |
| Guanosine | 284.2 | 152.1 | 16 | 180 | 10 | 16 | 15 |
| Inosine | 269.2 | 137.1 | 11 | 150 | 10 | 14 | 15 |
| 5-Methylcytidine | 258.3 | 126.1 | 22 | 200 | 10 | 16 | 15 |
| 5-Hydroxymethyl-2′-deoxyuridine | 259.2 | 143.1 | 17 | 160 | 14 | 12 | 12 |
| 8-Hydroxy-2′-deoxyguanosine | 284.2 | 168.1 | 19 | 170 | 12 | 12 | 10 |

Of the ten nucleosides investigated using HPLC/TQMS, eight were quantifiable. Of the eight metabolites that were quantified, three methylated (modified) and two major nucleosides were significantly different in the patient vs. control group.

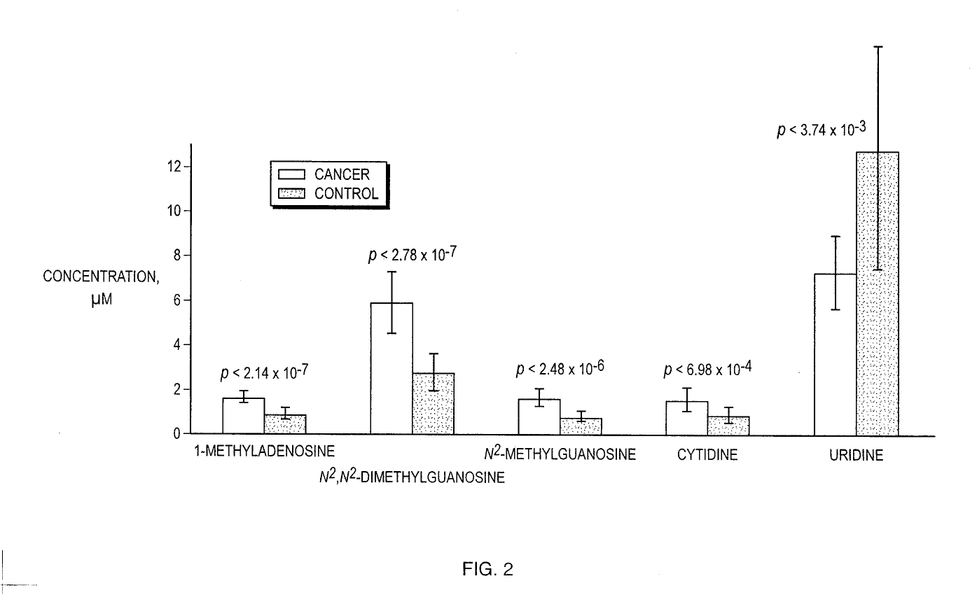

As shown in FIG. 2, in the serum samples from patients suffering from esophageal adenocarcinoma, 1-methyladenosine, N²,N²-dimethylguanosine, N²-methylguanosine and cytidine were significantly elevated, while uridine showed significantly lower concentration, compared to control serum samples. Student's t-test was utilized to evaluate the difference between patient and control groups. A p-value<0.05 was regarded as significant. The metabolic levels of 1-methyladenosine (p<2.14×10⁻⁷), N²,N²-dimethylguanosine (p<2.78×10⁻⁷), N²-methylguanosine (p<2.48×10⁻⁶) and cytidine (p<6.98×10⁻⁴) were significantly elevated while the concentration of uridine (p<3.74×10⁻³) was significantly lowered in serum samples from EAC patients compared to those of a control group of subjects.

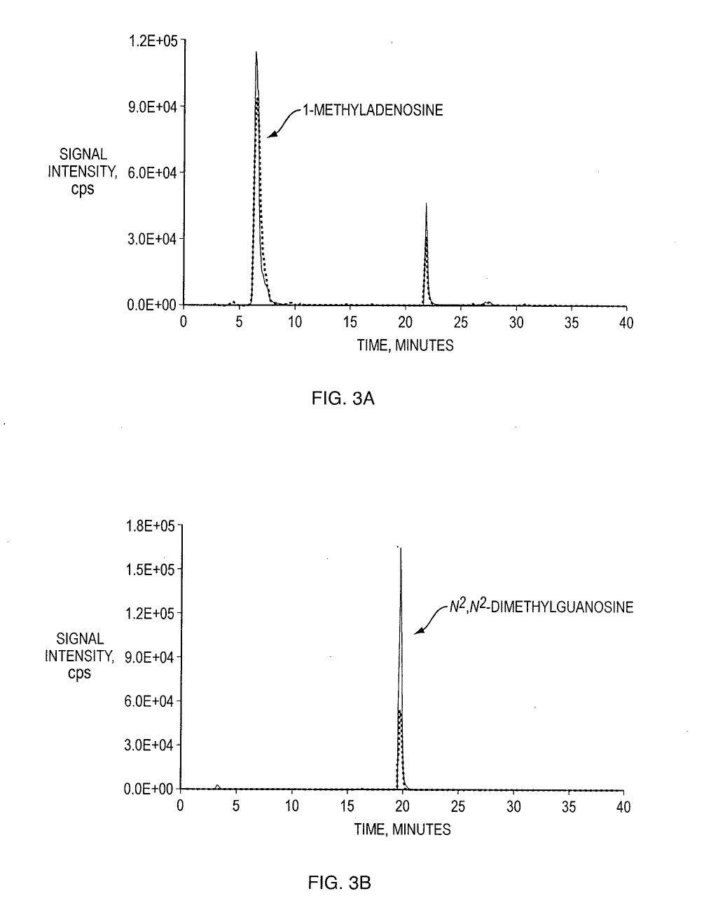

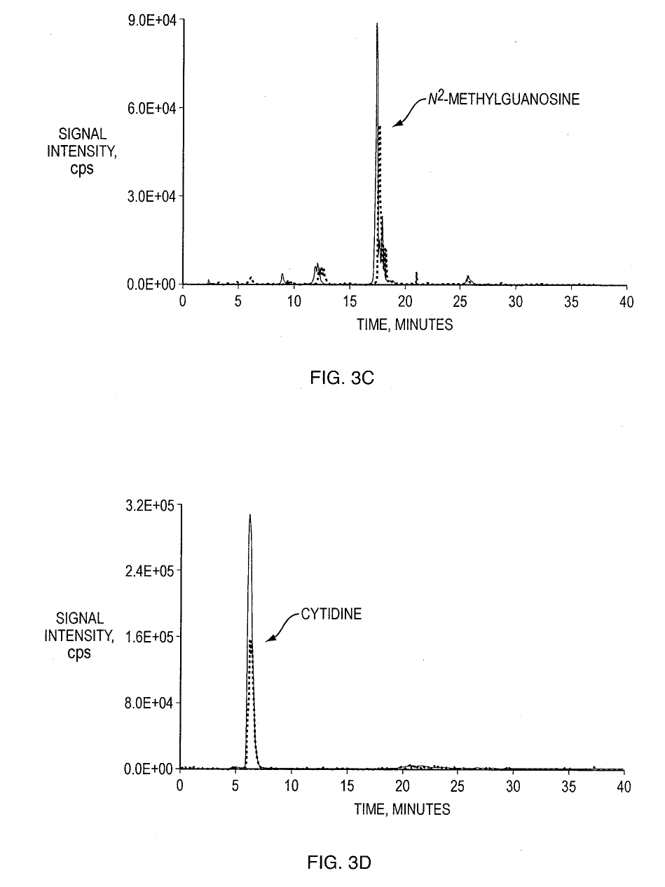

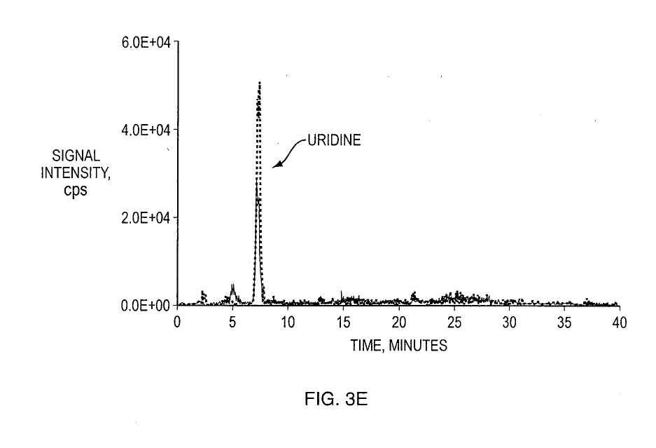

FIG. 3A-FIG. 3E show that good signal-to-noise ratios and well isolated MRM peaks were observed in the analysis. FIG. 3A-FIG. 3E is a series of HPLC-TQMS chromatograms of 1-methyladenosine (FIG. 3A), N², N²-dimethylguanosine (FIG. 3B), N²-methylguanosine (FIG. 3C), cytidine (FIG. 3D) and uridine (FIG. 3E) comparing results from one serum samples from an EAC patient (continuous line) and one serum samples from a control subjects (dashed line). The EAC sample had higher concentration of each of the nucleosides except uridine. Some of the precursor/fragment ion transition pairs produced multiple peaks including 1-methyladenosine, N²-methylguanosine. These secondary peaks were the product of identical MRM transitions from isomeric analogues of these metabolites. In the preliminary studies, a few control serum samples were spiked with the standard nucleosides in order to confirm the identity of the primary ion-pair transitions.

Although the 14 esophageal adenocarcinoma samples were classified in 4 different clinical stages, the number of samples within each stage was insufficient to investigate the correlation between the metabolic levels and disease progression. However, it is worth mentioning that among all EAC samples, the three serum specimens from the patients with metastatic carcinoma had the highest concentration of N²,N²-dimethylguanosine and 1-methyl-adenosine. The levels of the other three nucleosides did not show any recognizable pattern.

We were unable to quantify the metabolic levels of two methylated nucleosides. The MRM transitions of 5-hydroxymethyl-2′-deoxyuridine (259.2/143.1) and 8-hydroxy-2′-deoxy-guanosine (284.2/168.1) were present in fewer than half of the EAC and control samples, so we could not reliably determine the metabolic levels of these two metabolites. Interestingly, of all ten investigated metabolites, 5-hydroxymethyl-2′-deoxyuridine and 8-hydroxy-2′-deoxy-guanosine are the only molecules that, in their MRM transition, lose the 2′-deoxyribose moiety to form the [MH-116]+fragment ion. Since the 2′-deoxyribose sugar is found in DNA, and the ribose moiety is part of RNA, 5-hydroxymethyl-2′-deoxyuridine and 8-hydroxy-2′-deoxy-guanosine metabolites are products of DNA, rather than RNA turnover. The concentration of urinary 8-hydroxy-2′-deoxyguanosine was previously reported in breast and colorectal cancer patients as well as in rats exposed to bisphenol A-induced oxidative stress. An increased presence of urinary 5-hydroxymethyl-2′-deoxyuridine was reported in breast cancer samples and rats exposed to bisphenol A-induced oxidative stress, but in both studies the authors reported a positive MRM ion transition of 242/126. In our study, we monitored a positive ion transition of 259/143 based on the fact that the molecular mass of 5-hydroxymethyl-2′-deoxyuridine is 258 amu (atomic mass units).

The results show that the levels of three modified (1-methyladenosine, N²,N²-dimethyl-guanosine, N²-methylguanosine) nucleosides and one major (cytidine) nucleoside were significantly elevated in the EAC samples. This finding could indicate increased methylation of tRNA in esophageal adenocarcinoma patients similar to the DNA hypermethylation linked to carcinogenesis, in which the cytosine ring in the DNA chain undergoes excessive methylation at the carbon 5 position. The results show that in esophageal adenocarcinoma, hypermethylation of tRNA is expressed by excessive methylation at either the adenine ring nitrogen 1 position in case of 1-methyladenosine, or at the guanine ring nitrogen N² position in case of N²,N²-dimethyl-guanosine and N²-methylguanosine.

The level of uridine was significantly lower in EAC than in control samples. This may indicate that uridine is converted to pseudouridine at a faster rate in esophageal cancer patients than in healthy individuals. Pseudouridine was not quantified in the examples, which emphasized only nucleosides with fragmentation paths that generate [MH-132]⁺ and [MH-116]⁺ daughter ions. The ribose sugar and nitrogenous base in pseudouridine are not connected through the N-glycosidic bond as in uridine and other modified nucleosides, but through the more stable C-glycosidic bond. As a result, in the process of fragmentation pseudouridine will not lose the ribose moiety to form [MH-132]⁺ ion, but will have a more complex fragmentation pattern.

In order to eliminate the possibility that the concentrations of the nucleosides were age dependent and thus might separate cancer from normal based on age alone, the following analysis was performed. The cancer patient samples were split into two groups (patients younger and older than the average age of 65.4 years) and compared to the concentrations of the 5 nucleosides with low p-values in the EAC versus normal comparison. As shown in FIG. 4A-FIG. 4E, the concentrations of 1-methyladenosine, N²,N²-dimethylguanosine, N²-methyl-guanosine, cytidine and uridine were not significantly different between the two patient groups. P-values in this age-based comparison were respectively 0.35, 0.69, 0.67, 0.40, 0.74. Thus, the concentrations of these nucleosides in the EAC patients were not age-related.

In order to evaluate the reproducibility of the HPLC-TQMS method, two validation assays were performed under exactly the same experimental conditions that were used to quantify the nucleosides in the standard and serum samples. In the first assay, the quantitation of the eight metabolites was repeated in the EAC and control serum samples. The within-3-day coefficients of variance (CV) for the eight metabolites was determined. N²,N²-dimethyl-guanosine and uridine demonstrated the lowest within-3-day CV of ≦10.3% and ≦14.1%, respectively, whereas cytidine and guanosine exhibited the highest variations of ≦43.1% and ≦49.8%, correspondingly. The second validation assay was performed on a set of 3 standard mixtures containing each analyte at the concentrations of 0.200, 1.00 and 5.00 μM. For all eight metabolites, within-day variations were ≦19.0% and within-3-day maximum CVs were ≦21.7.

The method validation data for each quantified metabolite are listed in Table 2, below. Higher within-3-day variations in the serum samples compared to the standard mixtures could be expected because the whole set of biological samples (n=26 total) required a much longer acquisition time. The samples were kept in the autosampler at +4° C. throughout the whole analysis (˜48 h), exposing the nucleosides to potential cross-reactions and degradation. Nevertheless, the validation assay still showed that the metabolic levels of 1-methyladenosine, N²,N²-dimethylguanosine, N²-methylguanosine and cytidine were significantly elevated, while the concentration of uridine was significantly lower in the EAC specimens.

TABLE 2
METABOLIC LEVELS OF 8 MODIFIED NUCLEOSIDES IN ESOPHAGEAL ADENOCARCINOMA
PATIENTS AND CONTROL SAMPLES DETERMINED USING LC-TQMS.

| | Conc., EAC | Conc., Control | | CV, 3-Day, | CV, 1-Day, | CV, 3-Day, |
|---|---|---|---|---|---|---|
| COMPOUND | (μM) | (μM) | p-Value | Serum (%) | Std. Mix. (%) | Std. Mix. (%) |
| 1-Methyladenosine | 1.64 ± 0.28 | 0.89 ± 0.26 | 2.14 × 10⁻⁷ | ≦27.2 | ≦14.3 | ≦16.9 |
| N²,N²-Dimethylguanosine | 5.91 ± 1.35 | 2.77 ± 0.81 | 2.78 × 10⁻⁷ | ≦10.3 | ≦3.30 | ≦5.20 |
| N²-Methylguanosine | 1.63 ± 0.43 | 0.77 ± 0.22 | 2.48 × 10⁻⁶ | ≦20.4 | ≦13.7 | ≦19.0 |
| Cytidine | 1.58 ± 0.54 | 0.87 ± 0.37 | 6.98 × 10⁻⁴ | ≦43.1 | ≦18.9 | ≦17.3 |
| Uridine | 7.30 ± 1.65 | 12.80 ± 5.18 | 3.74 × 10⁻³ | ≦14.1 | ≦5.00 | ≦11.3 |
| 5-Methylcytidine | 1.02 ± 0.75 | 0.60 ± 0.27 | 6.90 × 10⁻² | ≦33.6 | ≦14.6 | ≦19.9 |
| Inosine | 17.95 ± 18.29 | 9.99 ± 8.57 | 1.62 × 10⁻¹ | ≦37.5 | ≦11.2 | ≦17.4 |
| Guanosine | 3.64 ± 1.53 | 3.52 ± 1.68 | 8.50 × 10⁻¹ | ≦49.8 | ≦19.0 | ≦21.7 |

SPE sample recovery measurements were made for the five nucleosides identified as putative esophageal adenocarcinoma biomarkers. Standard mixtures of these five nucleosides were made at the concentrations of 0.200 μM, 1.00 μM and 5.00 μM. The standard samples were dissolved in 5 mM aqueous ammonium acetate, and the pH of the solvent was adjusted to 7.4 with acetic acid. SPE was performed for each of the standard mixtures following the SPE procedure described above. After SPE, each dry sample was reconstituted in 1.00 mL of 5 mM aqueous ammonium acetate. At the end, 10.0 μL of each standard mixture (prior to SPE) and reconstituted sample (after SPE) were analyzed. Prior to the LC-TQMS analysis, 10.0 μL of 50.0 μM iSTD was added to 1.00 mL of each sample. By comparing the MRM data obtained from the samples prior to and after SPE, we determined that the sample recovery for the five nucleosides was the lowest for N²-methylguanosine (89.1±3.53%) and highest for 1-methyl-adenosine (94.3±0.92%).

The results identified five biomarkers for esophageal adenocarcinoma. It was determined that the metabolic levels of 1-methyladenosine, N²,N²-dimethylguanosine, N²-methylguanosine and cytidine were significantly increased while the concentration of uridine was significantly lower in EAC compared to control serum samples.

The elevated presence of 1-methyladenosine, N²,N²-dimethylguanosine and N²-methyl-guanosine is an indicator of hypermethylation of tRNA associated with esophageal carcinoma that occurs at either the adenine ring nitrogen 1 position or at the guanine ring nitrogen N position. The level of uridine was significantly decreased in EAC patients compared to the control group, which in turn could potentially indicate that uridine is converted to pseudouridine at a higher rate in esophageal adenocarcinoma patients than in healthy individuals. Among 14 EAC samples, the three specimens from the patients with metastatic malignancy had the highest concentration of N²,N²-dimethylguanosine and 1-methyladenosine.

While an exemplary embodiment incorporating the principles of the present disclosure has been disclosed hereinabove, the present disclosure is not limited to the disclosed embodiments. Instead, this application is intended to cover any variations, uses, or adaptations of the disclosure using its general principles. Further, this application is intended to cover such departures from the present disclosure as come within known or customary practice in the art to which this disclosure pertains and which fall within the limits of the appended claims.

While the foregoing invention has been described in some detail for purposes of clarity and understanding, these particular embodiments are to be considered as illustrative and not restrictive. It will be appreciated by one skilled in the art from a reading of this disclosure that various changes in form and detail can be made without departing from the true scope of the invention, which is to be defined by the appended claims rather than by the specific embodiments.

All listed patents, patent publications and non-patent literature are incorporated herein by reference. In the case of inconsistencies, the present disclosure, including definitions, will control.

What is claimed is:

1. A method for determining the esophageal cancer status of a patient, comprising:
determining the presence and concentration within a biofluid sample from the patient of at least one compound selected from the group consisting of 1-methyladenosine, N²,N²-dimethylguanosine, N²-methylguanosine, cytidine, uridine and combinations thereof; and
correlating the measured concentration of the metabolite species with an esophageal cancer status.

2. The method of claim 1 wherein the combination of metabolite species is selected from the group consisting of
a. 1-methyladenosine and N²,N²-dimethylguanosine;
b. 1-methyladenosine, N²,N²-dimethylguanosine, N²-methylguanosine, cytidine, and uridine;
c. 1-methyladenosine, N²,N²-dimethylguanosine, and N²-methylguanosine;
d. 1-methyladenosine, N²,N²-dimethylguanosine, N²-methylguanosine, and uridine;
e. 1-methyladenosine, N²,N²-dimethylguanosine, N²-methylguanosine, and cytidine;
f. 1-methyladenosine, N²,N²-dimethylguanosine, and uridine;
g. N²,N²-dimethylguanosine, N²-methylguanosine, cytidine, and uridine; and
h. N²,N²-dimethylguanosine, N²-methylguanosine, and uridine.

3. The method of claim 1 wherein the esophageal cancer status is normal, Barrett's esophagus, high-grade dysplasia or esophageal adenocarcinoma.

4. The method of claim 1 wherein the biofluid is blood.

5. The method of claim 1 wherein the biofluid is serum.

6. The method of claim 1 wherein the at least one metabolite species are adapted to function as biomarkers.

7. A biomarker for detecting esophageal cancer, comprising at least one metabolite species or parts thereof, selected from the group consisting of 1-methyladenosine, N²,N²-dimethylguanosine, N²-methylguanosine, cytidine, uridine and combinations thereof.

8. The biomarker of claim 7 wherein the combination of metabolite species is selected from the group consisting of
a. 1-methyladenosine and N²,N²-dimethylguanosine;
b. 1-methyladenosine, N²,N²-dimethylguanosine, N²-methylguanosine, cytidine, and uridine;
c. 1-methyladenosine, N²,N²-dimethylguanosine, and N²-methylguanosine;
d. 1-methyladenosine, N²,N²-dimethylguanosine, N²-methylguanosine, and uridine;
e. 1-methyladenosine, N²,N²-dimethylguanosine, N²-methylguanosine, and cytidine;
f. 1-methyladenosine, N²,N²-dimethylguanosine, and uridine;
g. N²,N²-dimethylguanosine, N²-methylguanosine, cytidine, and uridine; and
h. N²,N²-dimethylguanosine, N²-methylguanosine, and uridine.

9. The biomarker of claim 7 wherein the biomarker is contained in a biochemically compatible solution.

10. The biomarker of claim 7 wherein the biomarker is contained in a compatible buffered aqueous solution.

11. A kit for the analysis of a sample of a biofluid of a subject, comprising:
a. standard aliquots of 1-methyladenosine, N²,N²-dimethylguanosine, N²-methyl-guanosine, cytidine, and uridine;
b. an aliquot of an internal standard; and
c. an aliquot of a control biofluid.

12. The kit of claim 11 wherein the biofluid is serum.

13. The kit of claim 11 wherein the internal standard is 7-deazaadenosine.

14. The kit of claim 11 further comprising instructions for use.
